SEQUENCE LISTING (14 sequences)

SEQ ID NO: 1 - DNA, 100 bp, Zaire ebolavirus
  misc_feature (1)..(100): Ebola virus, Zaire strain, the 5 terminal segment
cggacacacaaaaagaaagaagaatttttaggatcttttgtgtgcgaataactatgaggaagattaataattttcctctcattgaaatttatatcggaat

SEQ ID NO: 2 - DNA, 100 bp, Bundibugyo ebolavirus
  misc_feature (1)..(100): Ebola virus, Bundibugyo strain, the 5 terminal segment
cggacacacaaaaagaatgaaggattttgaatctttattgtgtgcgagtaactacgaggaagattaaagattttcctctcattgaaattgaaattgagat

SEQ ID NO: 3 - DNA, 100 bp, Tai Forest ebolavirus
  misc_feature (1)..(100): Ebola virus, Tai Forest strain, the 5 terminal segment
cggacacacaaaaagaaagaaggttttttgatctttattgtgtgcgaataactatgaggaagattaataattttcctctcattgacacttacattaagat

SEQ ID NO: 4 - DNA, 100 bp, Sudan ebolavirus
  misc_feature (1)..(100): Ebola virus, Sudan strain, the 5 terminal segment
cggacacacaaaaagaaagaaaagtttttnagactttttgtgtgcgaataactatgaggaagattaatcattttcctcaaactcaaactaatattnacat

SEQ ID NO: 5 - DNA, 100 bp, Reston ebolavirus
  misc_feature (1)..(100): Ebola virus, Reston strain, the 5 terminal segment
cggacacacaaaaagaaaaaagnttttttaagantttttgtgtncgagtaactatgaggaagattaacagttntcctcagtttaagntatacactgaaat

SEQ ID NO: 6 - DNA, 17 bp, Artificial Sequence
  Synthetic  5 terminal segment forward primer
gacacacaaaaagaawg

SEQ ID NO: 7 - DNA, 20 bp, Artificial Sequence
  Synthetic  5 terminal segment reverse primer
ngaggaaaatvnttaatctt

SEQ ID NO: 8 - DNA, 22 bp, Artificial Sequence
  Synthetic  5 terminal segment probe
ttgtgtgcgartaactaygagg

SEQ ID NO: 9 - DNA, 100 bp, Artificial Sequence
  Consensus sequence derived from various Ebola strains
cggacacacaaaaagaawraarndttttknrkmnttwttgtgtncgartaactaygaggaagattaahvrttntcctcwvwytvavnywdahayynrvat

SEQ ID NO: 10 - DNA, 100 bp, Artificial Sequence
  Consensus sequence derived from various Ebola strains
cggacacacaaaaagaawgaarrdttttknrkmyytwttgtgtgcgartaactaygaggaagattaawvattttcctcwmaytsamaytdahatynrvat

SEQ ID NO: 11 - DNA, 100 bp, Artificial Sequence
  Consensus sequence derived from various Ebola strains
cggacacacaaaaagaawgaagrwttttkrrkmtytwttgtgtgcgartaactaygaggaagattaawrattttcctctcattgamaytkahatyrrrat

SEQ ID NO: 12 - DNA, 100 bp, Artificial Sequence
  Consensus sequence derived from various Ebola strains
cggacacacaaaaagaawgaaggwttttkratctttattgtgtgcgartaactaygaggaagattaawrattttcctctcattgamaytkamattragat

SEQ ID NO: 13 - DNA, 100 bp, Artificial Sequence
  Consensus sequence derived from various Ebola strains
cggacacacaaaaagaaagaarartttttnrgayyttttgtgtgcgaataactatgaggaagattaatmattttcctcwmaytsaaaytwatatynrmat

SEQ ID NO: 14 - DNA, 100 bp, Artificial Sequence
  Consensus sequence derived from various Ebola strains
cggacacacaaaaagaawgaarrdttttknrkkmytwttgtgtgcgartaactakgaggaagattaawvattttcctcwmaytsamaytdahatynrvat

(19) United States
(12) Patent Application Publication
(10) Pub. No.: US 2016/0122834 A1
(43) Pub. Date: May 5, 2016

(54) METHODS OF DETECTING EBOLA

(71) Applicant: Cepheid, Sunnyvale, CA (US)

(72) Inventor: Robert Jones, Seattle, WA (US)

(21) Appl. No.: 14/887,609

(22) Filed: Oct. 20, 2015

Related U.S. Application Data
(60) Provisional application No. 62/072,790, filed on Oct. 30, 2014.

(51) Int. Cl. C12Q 1/70

(52) U.S. Cl. CPC C12Q 1/701

(57) ABSTRACT

Compositions and methods for detecting Ebola are provided.

### 1. CROSS-REFERENCE TO RELATED APPLICATIONS

This application claims the benefit of priority of U.S. Provisional Application No. 62/072,790, filed Oct. 30, 2014, which is incorporated by reference herein in its entirety for any purpose.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “01148-0013-00US_ST25.txt” created on Oct. 27, 2015 and is 6,099 bytes in size. The sequence listing contained in this .txt file is part of the specification and is hereby incorporated by reference herein in its entirety.

### 2. FIELD OF THE INVENTION

Compositions and methods for detecting the Ebola virus are provided. In particular, Ebola markers and panels of markers useful in the detection of Ebola are provided.

### 3. BACKGROUND

Ebola virus (“Ebola”) causes a severe disease called Ebola Virus Disease (EVD) that includes vomiting, diarrhea, abdominal pain and internal and external bleeding, eventually causing death in up to 90% of those infected. There are several strains of Ebola virus that can cause EVD, including Zaire Ebola virus (EBOV or EBOV-Z), Bundibugyo virus (BDBV), Sudan virus (SUDV), Cote d′Ivoire Ebola Virus, and Tai Forest virus (TAFV). Most outbreaks have historically been caused by the Zaire strain. Ebola virus is a member of the Filovirus family, and is a single-stranded, negative-sense RNA virus, with a genome of approximately 19,000 nucleotides, encoding seven proteins that include nucleoprotein (NP), polymerase cofactor VP35, VP40, GP, transcription activator VP30, VP24, and RNA polymerase (L). The 3′ “leader” and 5′ “trailer” regions are non-transcribed.

### 4. SUMMARY

In some embodiments, methods of detecting the presence or absence of the Ebola virus (“Ebola”) in a sample from a subject are provided. In some embodiments, a method comprises detecting the presence or absence of the 5′ terminal segment of the Ebola virus in the sample. In some embodiments, methods of determining whether a subject has Ebola are provided. In some embodiments, a method comprises detecting the presence or absence of of the 5′ terminal segment of the Ebola virus in a sample from the subject.

In some embodiments, the Ebola virus is a subtype selected from Zaire, Bundibugyo, Cote D'Ivoire, Tai Forest, Sudan, and Reston. In some embodiments, the Ebola virus is a subtype selected from Zaire, Bundibugyo, Tai Forest, and Sudan.

In some embodiments, the 5′ terminal segment of the Ebola virus is nucleotides 1 to 100. In some embodiments, the 5′ terminal segment of the Ebola virus is at least 80%, at least 85%, at least 90% identical to a sequence selected from SEQ ID NOs: 1 to 5. In some embodiments, the 5′ terminal segment of the Ebola virus is at least 80%, at least 85%, at least 90% identical to a sequence selected from SEQ ID NOs: 1 to 4. In some embodiments, the 5′ terminal segment of the Ebola virus is nucleotides 1 to 90. In some embodiments, the 5′ terminal segment of the Ebola virus is at least 80%, at least 85%, at least 90% identical to nucleotides 1 to 90 of a sequence selected from SEQ ID NOs: 1 to 5. In some embodiments, the 5′ terminal segment of the Ebola virus is at least 80%, at least 85%, at least 90% identical to nucleotides 1 to 90 of a sequence selected from SEQ ID NOs: 1 to 4. In some embodiments, the 5′ terminal segment of the Ebola virus is nucleotides 1 to 80. In some embodiments, the 5′ terminal segment of the Ebola virus is at least 80%, at least 85%, at least 90% identical to nucleotides 1 to 80 of a sequence selected from SEQ ID NOs: 1 to 5. In some embodiments, the 5′ terminal segment of the Ebola virus is at least 80%, at least 85%, at least 90% identical to nucleotides 1 to 80 of a sequence selected from SEQ ID NOs: 1 to 4.

In some embodiments, detection of the presence of any one of the Ebola genes indicates the presence of Ebola in the sample. In some embodiments, the subject has one or more symptoms of Ebola. In some embodiments, the subject has one or more symptoms selected from fever, muscle ache, headache, fatigue, vomiting, diarrhea, abdominal pain, and unexplained hemorrhaging.

In some embodiments, the method comprises detecting an exogenous control. In some embodiments, the exogenous control is a sample processing control. In some embodiments, the exogenous control comprises an RNA sequence that is not expected to be present in the sample. In some embodiments, the exogenous control is an Armored® RNA.

In some embodiments, the method comprises detecting an endogenous control. In some embodiments, the endogenous control is a sample adequacy control. In some embodiments, the endogenous control is selected from ABL mRNA, GUSB mRNA, GAPDH mRNA, TUBB mRNA, and UPK1a mRNA. In some embodiments, the endogenous control is ABL mRNA.

In some embodiments, the method comprises PCR. In some embodiments, the method comprises quantitative PCR. In some embodiments, the PCR reaction takes less than 2 hours, less than 90 minutes, or less than 1 hour from an initial denaturation step through a final extension step.

In some embodiments, the method comprises contacting nucleic acids from the sample with a first primer pair for detecting the 5′ terminal segment of the Ebola virus. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of a sequence selected from SEQ ID NOs: 1 to 5, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of a sequence selected from SEQ ID NOs: 1 to 5. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of nucleotides 1 to 23 of a sequence selected from SEQ ID NOs: 1 to 5, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of nucleotides 36 to 79 of a sequence selected from SEQ ID NOs: 1 to 5. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of nucleotides 1 to 23 of a sequence selected from SEQ ID NOs: 1 to 4, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of nucleotides 36 to 79 of a sequence selected from SEQ ID NOs: 1 to 4. In some embodiments, the first primer or the second primer or both the first primer and the second primer each comprises at least one degenerate nucleotide position, but no more than four degenerate nucleotide positions, or no more than three degenerate nucleotide positions. In some embodiments, each primer consists of 15 to 30 nucleotides. In some embodiments, the first primer has the sequence of SEQ ID NO: 6 and the second primer has the sequence of SEQ ID NO: 7.

In some embodiments, each primer pair produces an amplicon that is 50 to 100 nucleotides long, 50 to 90 nucleotides long, 50 to 80 nucleotides long, or 60 to 80 nucleotides long. In some embodiments, the method comprises forming an amplicon from each primer pair when the target of the primer pair is present. In some embodiments, the method comprises forming an Ebola 5′ terminal segment amplicon.

In some embodiments, the method comprises contacting the amplicons with an Ebola 5′ terminal segment probe. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of a sequence selected from SEQ ID NOs: 1 to 5. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides nucleotides 36 to 79 of a sequence selected from SEQ ID NOs: 1 to 5. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides nucleotides 36 to 79 of a sequence selected from SEQ ID NOs: 1 to 4. In some embodiments, the Ebola 5′ terminal segment probe comprises at least one degenerate nucleotide position. In some embodiments, the Ebola 5′ terminal segment probe has the sequence of SEQ ID NO: 8. In some embodiments, the Ebola 5′ terminal segment probe comprises a detectable label. In some embodiments, each probe comprises a fluorescent dye and a quencher molecule. In some embodiments, the probe consists of 15 to 30 nucleotides.

In some embodiments, the method comprises forming an exogenous control amplicon. In some embodiments, the method comprises contacting the exogenous control amplicon with a control probe capable of selectively hybridizing with the exogenous control amplicon. In some embodiments, the method comprises forming an endogenous control amplicon. In some embodiments, the method comprises contacting the endogenous control amplicon with a control probe capable of selectively hybridizing with the endogenous control amplicon. In some embodiments, the method comprises detecting the presence of absence of the 5′ terminal segment of the Ebola virus, and no other Ebola genes.

In some embodiments, the sample is selected from an oral swab sample and a blood sample.

In some embodiments, compositions are provided, comprising a first primer pair for detecting the 5′ terminal segment of the Ebola virus. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of a sequence selected from SEQ ID NOs: 1 to 5, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of a sequence selected from SEQ ID NOs: 1 to 5. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of nucleotides 1 to 23 of a sequence selected from SEQ ID NOs: 1 to 5, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of nucleotides 36 to 79 of a sequence selected from SEQ ID NOs: 1 to 5. In some embodiments, the first primer pair comprises a first primer and a second primer, wherein the first primer comprises a sequence that is at least 80%, at least 85%, at least 90%, at least 95%, or 100% identical to at least 15, at least 16, at least 17, at least 18, at least 19, at least 20, at least 21, at least 22, or at least 23 contiguous nucleotides of nucleotides 1 to 23 of a sequence selected from SEQ ID NOs: 1 to 4, and wherein the second primer comprises a sequence that is at least 80%, at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of nucleotides 36 to 79 of a sequence selected from SEQ ID NOs: 1 to 4. In some embodiments, the first primer or the second primer or both the first primer and the second primer each comprises at least one degenerate nucleotide position, but no more than four degenerate nucleotide positions, or no more than three degenerate nucleotide positions. In some embodiments, each primer consists of 15 to 30 nucleotides. In some embodiments, the first primer has the sequence of SEQ ID NO: 6 and the second primer has the sequence of SEQ ID NO: 7.

In some embodiments, the composition further comprises a primer pair for detecting an exogenous control. In some embodiments, the exogenous control is a sample processing control. In some embodiments, a composition further comprises a primer pair for detecting an endogenous control. In some embodiments, the exogenous control is a sample processing control and the endogenous control is a sample adequacy control.

In some embodiments, a composition further comprises an Ebola 5′ terminal segment probe. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of a sequence selected from SEQ ID NOs: 1 to 5. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides nucleotides 36 to 79 of a sequence selected from SEQ ID NOs: 1 to 5. In some embodiments, the Ebola 5′ terminal segment probe comprises a sequence that is at least 80%, at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides nucleotides 36 to 79 of a sequence selected from SEQ ID NOs: 1 to 4. In some embodiments, the Ebola 5′ terminal segment probe comprises at least one degenerate nucleotide position. In some embodiments, the Ebola 5′ terminal segment probe has the sequence of SEQ ID NO: 8.

In some embodiments, a composition further comprises a probe for detecting an exogenous control. In some embodiments, a composition further comprises a probe for detecting an endogenous control. In some embodiments, each probe comprises a detectable label. In some embodiments, the each probe comprises a fluorescent dye and a quencher molecule. In some embodiments, each probe consists of 15 to 30 nucleotides.

In some embodiments, the composition is a lyophilized composition. In some embodiments, the composition is in solution. In some embodiments, the composition comprises nucleic acids from a sample from a subject being tested for the presence of absence of Ebola. In some embodiments, the sample is selected from an oral swab sample and a blood sample.

In some embodiments, kits are provided, comprising any of the compositions described above. In some embodiments, a kit further comprises an exogenous control. In some embodiments, the exogenous control is an Armored® RNA. In some embodiments, the kit comprises dNTPs and/or a thermostable polymerase. In some embodiments, the kit comprises a reverse transcriptase.

In some embodiments, an oligonucleotide is provided, consisting of a sequence selected from SEQ ID NOs: 6 to 8, wherein the oligonucleotide comprises at least one modified nucleotide. In some embodiments, the oligonucleotide comprises a detectable label. In some embodiments, the oligonucleotide comprises a fluorescent dye and a quencher molecule. In some embodiments, the oligonucleotide is a fluorescence resonance energy transfer (FRET) probe. In some embodiments, a composition is provided, comprising at least one set of primers comprising a first primer consisting of the sequence of SEQ ID NO: 6 and a second primer consisting of the sequence of SEQ ID NO: 7, wherein at least one primer in the composition comprises at least one modified nucleotide.

In some embodiments, the composition comprises a probe consisting of the sequence of SEQ ID NOs: 8, wherein the probe comprises at least one modified nucleotide and/or a detectable label. In some embodiments, the composition comprises a first primer consisting of the sequence of SEQ ID NO: 6 and a second primer consisting of the sequence of SEQ ID NO: 7 and a probe consisting of SEQ ID NO: 8. In some embodiments, the probe is a fluorescence resonance energy transfer (FRET) probe. In some embodiments, the probe comprises at least one modified nucleotide. In some embodiments, the composition is a lyophilized composition. In some embodiments, the composition is in solution. In some embodiments, the composition comprises nucleic acids of a sample from a subject.

### 5. BRIEF DESCRIPTION OF FIGURE

FIG. 1A-1E show (A) an alignment of nucleotides 1 to 100 of five subtypes (or “strains”) of Ebola virus (SEQ ID NOS: 1-5, 9), (B) an alignment of nucleotides 1 to 100 of four subtypes of Ebola virus (SEQ ID NOS: 1-4, 10), (C) an alignment of nucleotides 1 to 100 of three subtypes of Ebola virus (SEQ ID NOS: 1-3, 11), (D) an alignment of nucleotides 1 to 100 of two subtypes of Ebola virus (SEQ ID NOS: 2-3, 12), and (E) an alignment of nucleotides 1 to 100 of two subtypes of Ebola virus (SEQ ID NOS: 1-4, 13).

FIG. 2 (SEQ ID NO: 14) shows an alternate consensus view of the consensus sequence from alignment of the four subtypes of the Ebola virus shown in FIG. 1B.

### 6. DETAILED DESCRIPTION

### 6.1. Definitions

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “detectably different” refers to a set of labels (such as dyes) that can be detected and distinguished simultaneously.

As used herein, the terms “patient” and “subject” are used interchangeably to refer to a human. In some embodiments, the methods described herein may be used on samples from non-human animals.

As used herein, the terms “oligonucleotide,” “polynucleotide,” “nucleic acid molecule,” and the like, refer to nucleic acid-containing molecules, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a single-stranded polynucleotide having fewer than 500 nucleotides. In some embodiments, an oligonucleotide is 8 to 200, 8 to 100, 12 to 200, 12 to 100, 12 to 75, or 12 to 50 nucleotides long. Oligonucleotides may be referred to by their length, for example, a 24 residue oligonucleotide may be referred to as a “24-mer.”

As used herein, the term “complementary” to a target RNA (or target region thereof), and the percentage of “complementarity” of the probe sequence to that of the target RNA sequence is the percentage “identity” to the sequence of target RNA or to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and sequence of the target RNA or the reverse complement of the sequence of the target RNA that best aligns therewith. The percentage is calculated by counting the number of aligned bases that are identical as between the 2 sequences, dividing by the total number of contiguous nucleotides in the probe, and multiplying by 100. When the term “complementary” is used, the subject oligonucleotide is at least 90% complementary to the target molecule, unless indicated otherwise. In some embodiments, the subject oligonucleotide is at least 91%, at least 92%, at least 93%, at least 94%, at least 95%, at least 96%, at least 97%, at least 98%, at least 99%, or 100% complementary to the target molecule.

A “primer” or “probe” as used herein, refers to an oligonucleotide that comprises a region that is complementary to a sequence of at least 8 contiguous nucleotides of a target nucleic acid molecule, such as DNA (e.g., a target gene) or an mRNA (or a DNA reverse-transcribed from an mRNA). In some embodiments, a primer or probe comprises a region that is complementary to a sequence of at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of a target molecule. When a primer or probe comprises a region that is “complementary to at least x contiguous nucleotides of a target molecule,” the primer or probe is at least 95% complementary to at least x contiguous nucleotides of the target molecule. In some embodiments, the primer or probe is at least 96%, at least 97%, at least 98%, at least 99%, or 100% complementary to the target molecule.

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8, Cook et al., J Microbiol Methods. 2003 May; 53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

Unless otherwise indicated, the term “hybridize” is used herein refer to “specific hybridization” which is the binding, duplexing, or hybridizing of a nucleic acid molecule preferentially to a particular nucleotide sequence, in some embodiments, under stringent conditions. The term “stringent conditions” refers to conditions under which a probe will hybridize preferentially to its target sequence, and to a lesser extent to, or not at all to, other sequences. A “stringent hybridization” and “stringent hybridization wash conditions” in the context of nucleic acid hybridization (e.g., as in array, Southern, or Northern hybridization) are sequence-dependent and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions for filter hybridizations are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Dependency of hybridization stringency on buffer composition, temperature, and probe length are well known to those of skill in the art (see, e.g., Sambrook and Russell (2001) Molecular Cloning: A Laboratory Manual (3rd ed.) Vol. 1-3, Cold Spring Harbor Laboratory, Cold Spring Harbor Press, NY).

A “sample,” as used herein, includes various nasal samples, such as nasopharyngeal swab samples, nasal aspirate samples, nasal wash samples, and other types of human samples. In some embodiments, a nasal sample comprises a buffer, such as a preservative. Further nonlimiting exemplary samples include nasal swabs, oropharyngeal swabs, throat swabs, bronchoalveolar lavage samples, bronchial aspirates, bronchial washes, endotracheal aspirates, endotracheal washes, tracheal aspirates, nasal secretion samples, mucus samples, sputum samples, and lung tissue samples. In some embodiments, the sample comprises a buffer, such as a preservative.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA. Nonlimiting exemplary endogenous controls include ABL mRNA, GUSB mRNA, GAPDH mRNA, TUBB mRNA, and UPK1a mRNA. In some embodiments, an endogenous control, such as an SAC, is selected that can be detected in the same manner as the target RNA is detected and, in some embodiments, simultaneously with the target RNA.

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target RNA. In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target RNA is detected and, in some embodiments, simultaneously with the target RNA. In some embodiments, the exogenous control is an RNA. In some such embodiments, the exogenous control is an Armored RNA®, which comprises RNA packaged in a bacteriophage protective coat. See, e.g., WalkerPeach et al., Clin. Chem. 45:12: 2079-2085 (1999).

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine. Thus, where an RNA sequence includes “T”, one skilled in the art would understand that that position in the native RNA is likely a uracil.

In the present disclosure, “a sequence selected from” encompasses both “one sequence selected from” and “one or more sequences selected from.” Thus, when “a sequence selected from” is used, it is to be understood that one, or more than one, of the listed sequences may be chosen.

In the present disclosure, a method that comprises detecting a “a set of Ebola markers consisting of . . . ” involves detection of only the Ebola markers of the set, and not any further Ebola markers. The method may comprise additional components or steps, however, such as for detecting another pathogen and/or endogenous and/or exogenous controls. Similarly, a method or composition that comprises “a set of Ebola marker primer pairs consisting of” and/or “a set of Ebola marker probes consisting of” can include primer pairs and/or probes for only the Ebola markers of the set, and not for any other Ebola markers. The method or composition may comprise additional components, however, such as one or more primer pairs to detect another pathogen and/or endogenous control primer pairs and/or exogenous control primer pairs.

### 6.2. Detecting Ebola

The present inventors have developed a fast and sensitive assay for detecting Ebola. In some embodiments, the assay comprises detecting the 5′ terminal segment of the Ebola virus. In some embodiments, the present methods detect at least two Ebola strains selected from Zaire, Bundibugyo, Cote D'Ivoire, Tai Forest, and Sudan. The present assay relies on the polymerase chain reaction (PCR), and can be carried out in a substantially automated manner using a commercially available nucleic acid amplification system. Exemplary nonlimiting nucleic acid amplification systems that can be used to carry out the methods of the invention include the GeneXpert® system, a GeneXpert® Infinity system, and a Smartcycler System (Cepheid, Sunnyvale, Calif.). The present assay can be completed in under 3 hours, and in some embodiments, under 2 hours, and in some embodiments, under 90 minutes, and in some embodiments, under 1 hour, using an automated system, for example, the GeneXpert® system.

6.2.1. General Methods

Compositions and methods for detecting Ebola are provided. In some embodiments, the method comprises detecting the 5′ terminal segment of the Ebola virus.

In some embodiments, a method of detecting Ebola in a subject comprises detecting the presence of the 5′ terminal segment of the Ebola virus in a sample from the subject. In some embodiments, the sample is selected from a swab sample taken in the mouth (e.g., taken with a flocked swab) and a blood sample, such as a blood sample taken from a finger stick.

In some embodiments, a method of detecting Ebola further comprises detecting at least one endogenous control, such as a sample adequacy control (SAC). In some embodiments, a method of detecting Ebola further comprises detecting at least one exogenous control, such as a sample processing control (SPC). In some embodiments, the SPC is Armored® RNA.

In some embodiments, a method of detecting Ebola comprises detecting the the 5′ terminal segment of the Ebola virus in a sample. In some embodiments, a method of detecting Ebola further comprises detecting a sample processing control (SPC), such as an Armored® RNA. In some embodiments, a method of detecting Ebola further comprises detecting a sample adequacy control (SAC), such as ABL mRNA.

In the present disclosure, the terms “target RNA” and “target gene” are used interchangeably to refer to the 5′ terminal segment of the Ebola virus, and also to other Ebola genes, as well as to exogenous and/or endogenous controls. Thus, it is to be understood that when a discussion is presented in terms of a target gene, that discussion is specifically intended to encompass the 5′ terminal segment of the Ebola virus, other Ebola genes, any endogenous control(s) (e.g., SAC), and any exogenous control(s) (e.g., SPC). As used herein, the term “target gene” may be used to refer to non-coding segments of the Ebola virus.

In the present disclosure, the term “5′ terminal segment of the Ebola virus” refers to a segment at the 5′ terminus of the Ebola virus that consists of at most the first 100 nucleotides of the virus. In some embodiments, the 5′ terminal segment of the Ebola virus is nucleotides 1 to 100. In some embodiments, the 5′ terminal segment of the Ebola virus is nucleotides 1 to 90. In some embodiments, the 5′ terminal segment of the Ebola virus is nucleotides 1 to 80. In some embodiments, a 5′ terminal segment of the Ebola virus is at least 70%, at least 75%, at least 80%, at least 85%, or at least 90% identical to the sequence of any one of SEQ ID NOs: 1 to 4. In some embodiments, a 5′ terminal segment of the Ebola virus is at least 70%, at least 75%, at least 80%, at least 85%, or at least 90% identical to nucleotides 1 to 90 of any one of SEQ ID NOs: 1 to 4. In some embodiments, a 5′ terminal segment of the Ebola virus is at least 70%, at least 75%, at least 80%, at least 85%, or at least 90% identical to nucleotides 1 to 80 of any one of SEQ ID NOs: 1 to 4.

In some embodiments, the presence of the 5′ terminal segment of the Ebola virus is detected in a swab sample taken from the mouth (an “oral swab sample”). In some embodiments, the target gene is detected in a blood sample. In some embodiments, a target gene is detected in a sample to which a buffer (such as a preservative) has been added. In some embodiments, the target gene is detected in an oral swab sample that has been placed in a buffer (such as a preservative).

In some embodiments, detection of the 5′ terminal segment of the Ebola virus in a sample from a subject indicates the presence of Ebola virus in the subject. In some embodiments, the detecting is done quantitatively. In other embodiments, the detecting is done qualitatively. In some embodiments, detecting a target gene comprises forming a complex comprising a polynucleotide and a nucleic acid selected from a target gene, a cDNA reverse transcribed from a target gene, a DNA amplicon of a target gene, and a complement of a target gene. In some embodiments, detecting a target gene comprises RT-PCR. In some embodiments, detecting a target gene comprises quantitative RT-PCR or real-time RT-PCR. In some embodiments, a sample adequacy control (SAC) and/or a sample processing control (SPC) is detected in the same assay as the target gene. In some embodiments, if the 5′ terminal segment of the Ebola virus is detected, Ebola is considered to be detected even if the SPC and/or SAC is not detected in the assay. In some embodiments, if the 5′ terminal segment of the Ebola virus is not detected, Ebola is considered to be not detected only if the SPC or SAC (or both, if both are included) is detected in the assay.

In some embodiments, the presence of the 5′ terminal segment of the Ebola virus can be measured in samples collected at one or more times from a subject to monitor a subject who is at risk of developing Ebola. In some embodiments, a subject who is at risk of developing Ebola is a subject who has come into contact with a person exhibiting symptoms of Ebola, or who has been diagnosed as having Ebola. In some embodiments, the presence of the 5′ terminal segment of the Ebola virus can be measured in samples collected at one or more times from a subject to monitor a subject who is being treated for Ebola. In some embodiments, the present assay may be used as part of routine and/or preventative healthcare for a subject, e.g., in areas in which Ebola is endemic.

In some embodiments, a sample to be tested is a blood sample (such as a blood sample taken by finger stick). In some embodiments, the blood sample is used directly in the methods described herein. In some embodiments, a buffer (such as a preservative) is added to the blood sample. In some embodiments, the buffer is added to the blood sample 5 minutes, within 10 minutes, within 30 minutes, within 1 hour, or within 2 hours of sample collection.

In some embodiments, a sample to be tested is an oral swab sample (such as a flocked swab sample taken in the mouth). In some embodiments, the swab is placed in a buffer. In some embodiments, the swab is immediately placed in the buffer. In some embodiments, the swab is placed in the buffer within 5 minutes, within 10 minutes, within 30 minutes, within 1 hour, or within 2 hours of sample collection.

In some embodiments, less than 5 ml, less than 4 ml, less than 3 ml, less than 2 ml, less than 1 ml, or less than 0.75 ml of sample or buffered sample are used in the present methods. In some embodiments, 0.05 ml to 1 ml of sample or buffered sample is used in the present methods. In some embodiments, the blood sample or oral swab is placed in 3 ml of buffer, and 1 ml of the buffered sample is used in an assay described herein.

In some embodiments, the sample to be tested is another bodily fluid, such as saliva, nasal swabs, oropharyngeal swabs, throat swabs, bronchoalveolar lavage samples, nasal secretion samples, mucus samples, sputum samples, fecal samples, etc.

The clinical sample to be tested is, in some embodiments, fresh (i.e., never frozen). In other embodiments, the sample is a frozen specimen. In some embodiments, the sample is a tissue sample, such as a formalin-fixed paraffin embedded sample. In some embodiments, the sample is a liquid cytology sample.

In some embodiments, the sample to be tested is obtained from an individual who has one or more symptoms of Ebola infection. Nonlimiting exemplary symptoms of Ebola include fever, headache, muscle pain, weakness or fatigue, vomiting, diarrhea, abdominal pain, unexplained hemorrhage (bleeding or bruising), and combinations of any of those symptoms. In some embodiments, the sample to be tested is obtained from an individual who is being treated for Ebola. In some embodiments, the sample to be tested is obtained from an individual who is being monitored for Ebola.

In some embodiments, methods described herein can be used for routine screening of healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

In some embodiments, the methods described herein can be used to assess the effectiveness of a treatment for Ebola infection in a patient.

In some embodiments, use of the 5′ terminal segment of the Ebola virus for detecting Ebola is provided.

In any of the embodiments described herein, the 5′ terminal segment of the Ebola virus may be detected in the same assay reaction as a sample processing control (SPC) and/or a sample adequacy control (SAC).

In some embodiments, a method of facilitating detection of Ebola in a subject is provided. Such methods comprise detecting the presence or absence of the 5′ terminal segment of the Ebola virus in a sample from the subject. In some embodiments, information concerning the presence or absence of the 5′ terminal segment of the Ebola virus in the sample from the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, detecting the presence or absence of the 5′ terminal segment of the Ebola virus is carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc.

In some embodiments, methods of detecting Ebola are provided. In some embodiments, methods of diagnosing Ebola infection are provided. In some embodiments, the method comprises obtaining a sample from a subject and providing the sample to a laboratory for detection of the the 5′ terminal segment of the Ebola virus in the sample. In some embodiments, the method further comprises receiving a communication from the laboratory that indicates the presence or absence of the 5′ terminal segment of the Ebola virus in the sample. A “laboratory,” as used herein, is any facility that detects the target gene in a sample by any method, including the methods described herein, and communicates the result to a medical practitioner. In some embodiments, a laboratory is under the control of a medical practitioner. In some embodiments, a laboratory is not under the control of the medical practitioner.

When a laboratory communicates the result of detecting the presence or absence of the 5′ terminal segment of the Ebola virus to a medical practitioner, in some embodiments, the laboratory indicates whether or not the the 5′ terminal segment of the Ebola virus was detected in the sample. In some embodiments, the laboratory indicates whether the sample comprises Ebola, by indicating, for example, “Ebola positive” or “Ebola negative” or “Ebola present” or “Ebola absent,” and the like.

As used herein, when a method relates to detecting Ebola, determining the presence of Ebola, monitoring for Ebola, and/or diagnosing Ebola infection, the method includes activities in which the steps of the method are carried out, but the result is negative for the presence of Ebola. That is, detecting, determining, monitoring, and diagnosing Ebola or Ebola infection include instances of carrying out the methods that result in either positive or negative results.

In some embodiments, at least one endogenous control (e.g., an SAC) and/or at least one exogenous control (e.g., an SPC) are detected simultaneously with the 5′ terminal segment of the Ebola virus in a single reaction. In some embodiments, at least one exogenous control (e.g., an SPC) is detected simultaneously with the 5′ terminal segment of the Ebola virus in a single reaction. In some embodiments, at least one endogenous control (e.g., an SAC) is detected simultaneously with the 5′ terminal segment of the Ebola virus in a single reaction. In some embodiments, at least one exogenous control (e.g., an SPC) and at least one endogenous control (e.g., an SAC) is detected simultaneously with the 5′ terminal segment of the Ebola virus in a single reaction.

In any of the embodiments described herein, the 5′ terminal segment of the Ebola virus may be detected along with one or more additional Ebola genes.

6.2.2. Exemplary Controls

In some embodiments, an assay described herein comprises detecting the 5′ terminal segment of the Ebola virus and at least one endogenous control. In some embodiments, the endogenous control is a sample adequacy control (SAC). In some such embodiments, if the 5′ terminal segment of the Ebola virus is not detected in a sample, and the SAC is also not detected in the sample, the assay result is considered “invalid” because the sample may have been insufficient. While not intending to be bound by any particular theory, an insufficient sample may be too dilute, contain too little cellular material, contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPK1a mRNA.

In some embodiments, an assay described herein comprises detecting the 5′ terminal segment of the Ebola virus and at least one exogenous control. In some embodiments, the exogenous control is a sample processing control (SPC). In some such embodiments, if the 5′ terminal segment of the Ebola virus is not detected in a sample, and the SPC is also not detected in the sample, the assay result is considered “invalid” because there may have been an error in sample processing, including but not limited to, failure of the assay. Nonlimiting exemplary errors in sample processing include, inadequate sample processing, the presence of an assay inhibitor, the presence of a nuclease (such as an RNase), compromised reagents, etc. In some embodiments, an exogenous control (such as an SPC) is added to a sample. In some embodiments, an exogenous control (such as an SPC) is added during performance of an assay, such as with one or more buffers or reagents. In some embodiments, when a GeneXpert® system is to be used, the SPC is included in the GeneXpert® cartridge. In some embodiments, an exogenous control (such as an SPC) is an Armored RNA®, which is protected by a bacteriophage coat.

In some embodiments, an endogenous control and/or an exogenous control is detected contemporaneously, such as in the same assay, as detection of the 5′ terminal segment of the Ebola virus. In some embodiments, an assay comprises reagents for detecting the 5′ terminal segment of the Ebola virus and an exogenous control simultaneously in the same assay reaction. In some such embodiments, for example, an assay reaction comprises a primer set for amplifying the 5′ terminal segment of the Ebola virus, and, a primer set for amplifying an exogenous control, and labeled probes for detecting the amplification products (such as, for example, TagMan® probes).

6.2.3. Exemplary Sample Preparation

6.2.3.1. Exemplary Buffers

In some embodiments, a buffer is added to the sample. In some embodiments, the buffer is added within one hour, two hours, three hours, or six hours of the time the sample was collected. In some embodiments, a buffer is added to the sample within one hour, two hours, three hours, or six hours before the sample is analyzed by the methods described herein.

In some embodiments, a swab sample is placed in a buffer. In some embodiments, the swab sample is placed in the buffer within one hour, two hours, three hours, or six hours of the time the swab sample was collected. In some embodiments, the swab sample is placed in a buffer within one hour, two hours, three hours, or six hours before the sample is analyzed by the methods described herein.

Non-limiting exemplary commercial buffers include the viral transport medium provided with the GeneXpert® Nasal Pharyngeal Collection Kit (Cepheid, Sunnyvale, Calif.); universal transport medium (UTM™, Copan, Murrieta, Calif.); universal viral transport medium (UVT, BD, Franklin Lakes, N.J.); M4, M4RT, M5, and M6 (Thermo Scientific). Further nonlimiting exemplary buffers include liquid Amies medium, PBS/0.5% BSA, PBS/0.5% gelatin, Bartel BiraTrans™ medium, EMEM, PBS, EMEM/1% BSA, sucrose phosphate, Trypticase™ soy broth (with or without 0.5% gelatin or 0.5% BSA), modified Stuart's medium, veal infusion broth (with or without 0.5% BSA), and saline.

6.2.3.2. Exemplary RNA Preparation

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen), Total RNA Extraction Kit (iNtRON Biotechnology), Total RNA Purification Kit (Norgen Biotek Corp.), RNAqueous™ (Ambion), MagMAX™ (Ambion), RecoverAll™ (Ambion), RNAeasy (Qiagen), etc.

In some embodiments, RNA levels are measured in a sample in which RNA has not first been purified from the cells. In some such embodiments, the cells are subject to a lysis step to release the RNA. Nonlimiting exemplary lysis methods include sonication (for example, for 2-15 seconds, 8-18 μm at 36 kHz); chemical lysis, for example, using a detergent; and various commercially available lysis reagents (such as RNAeasy lysis buffer, Qiagen). In some embodiments, RNA levels are measured in a sample in which RNA has been isolated.

In some embodiments, RNA is modified before a target RNA is detected. In some embodiments, all of the RNA in the sample is modified. In some embodiments, just the particular target RNAs to be analyzed are modified, e.g., in a sequence-specific manner. In some embodiments, RNA is reverse transcribed. In some such embodiments, RNA is reverse transcribed using MMLV reverse transcriptase. Nonlimiting exemplary conditions for reverse transcribing RNA using MMLV reverse transcriptase include incubation from 5 to 20 minutes at 40° C. to 50° C.

When a target RNA is reverse transcribed, a DNA complement of the target RNA is formed. In some embodiments, the complement of a target RNA is detected rather than a target RNA itself (or a DNA copy of the RNA itself). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In some such embodiments, a polynucleotide for detection comprises at least a portion that is identical in sequence to the target RNA, although it may contain thymidine in place of uridine, and/or comprise other modified nucleotides.

6.2.4. Exemplary Analytical Methods

As described above, methods are presented for detecting Ebola. The methods comprise detecting the presence of the 5′ terminal segment of the Ebola virus in a sample from a subject. In some embodiments, the method further comprises detecting one or more additional target genes. In some embodiments, the method further comprises detecting at least one exogenous control (such as an SPC) and/or at least one endogenous control (such as an SAC). In some embodiments, detection of the 5′ terminal segment of the Ebola virus indicates the presence of Ebola, even if the endogenous control and/or exogenous control is not detected in the assay. In some embodiments, if the 5′ terminal segment of the Ebola virus is not detected, the result is considered to be negative for Ebola only if the control(s) are detected.

Any analytical procedure capable of permitting specific detection of a target gene may be used in the methods herein presented. Exemplary nonlimiting analytical procedures include, but are not limited to, nucleic acid amplification methods, PCR methods, isothermal amplification methods, and other analytical detection methods known to those skilled in the art.

In some embodiments, the method of detecting a target gene, such as the 5′ terminal segment of the Ebola virus, comprises amplifying the gene and/or a complement thereof. Such amplification can be accomplished by any method. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany.

When a target gene is amplified, in some embodiments, an amplicon of the target gene is formed. An amplicon may be single stranded or double-stranded. In some embodiments, when an amplicon is single-stranded, the sequence of the amplicon is related to the target gene in either the sense or antisense orientation. In some embodiments, an amplicon of a target gene is detected rather than the target gene itself. Thus, when the methods discussed herein indicate that a target gene is detected, such detection may be carried out on an amplicon of the target gene instead of, or in addition to, the target gene itself. In some embodiments, when the amplicon of the target gene is detected rather than the target gene, a polynucleotide for detection is used that is complementary to the complement of the target gene. In some embodiments, when the amplicon of the target gene is detected rather than the target gene, a polynucleotide for detection is used that is complementary to the target gene. Further, in some embodiments, multiple polynucleotides for detection may be used, and some polynucleotides may be complementary to the target gene and some polynucleotides may be complementary to the complement of the target gene.

In some embodiments, the method of detecting a target gene, such as the 5′ terminal segment of the Ebola virus, comprises PCR, as described below. In some embodiments, detecting one or more target genes comprises real-time monitoring of a PCR reaction, which can be accomplished by any method. Such methods include, but are not limited to, the use of TaqMan®, molecular beacons, or Scorpion probes (i.e., energy transfer (ET) probes, such as FRET probes) and the use of intercalating dyes, such as SYBR green, EvaGreen, thiazole orange, YO-PRO, TO-PRO, etc.

Nonlimiting exemplary conditions for amplifying a cDNA that has been reverse transcribed from the target RNA are as follows. An exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot start function. In some embodiments, the amplification reaction occurs in a GeneXpert® cartridge, and amplification of the target genes and an exogenous control occurs in the same reaction. In some embodiments, detection of the target genes occurs in less than 3 hours, less than 2.5 hours, less than 2 hours, less than 1 hour, or less than 30 minutes from initial denaturation through the last extension.

In some embodiments, detection of a target gene comprises forming a complex comprising a polynucleotide that is complementary to a target gene or to a complement thereof, and a nucleic acid selected from the target gene, a DNA amplicon of the target gene, and a complement of the target gene. Thus, in some embodiments, the polynucleotide forms a complex with a target gene. In some embodiments, the polynucleotide forms a complex with a complement of the target RNA, such as a cDNA that has been reverse transcribed from the target RNA. In some embodiments, the polynucleotide forms a complex with a DNA amplicon of the target gene. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target gene, complement of the target gene, or DNA amplicon of the target gene. The polynucleotide, in some embodiments, is a primer or probe.

In some embodiments, a method comprises detecting the complex. In some embodiments, the complex does not have to be associated at the time of detection. That is, in some embodiments, a complex is formed, the complex is then dissociated or destroyed in some manner, and components from the complex are detected. An example of such a system is a TagMan® assay. In some embodiments, when the polynucleotide is a primer, detection of the complex may comprise amplification of the target gene, a complement of the target gene, or a DNA amplicon of the target gene.

In some embodiments the analytical method used for detecting at least one target gene in the methods set forth herein includes real-time quantitative PCR. In some embodiments, the analytical method used for detecting at least one target gene includes the use of a TagMan® probe. The assay uses energy transfer (“ET”), such as fluorescence resonance energy transfer (“FRET”), to detect and quantitate the synthesized PCR product. Typically, the TagMan® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TagMan® probe is bound, the 5′-nuclease of the polymerase cleaves the probe, decoupling the dye and the quencher so that the dye signal (such as fluorescence) is detected. Signal (such as fluorescence) increases with each PCR cycle proportionally to the amount of probe that is cleaved.

In some embodiments, a target gene is considered to be detected if any signal is generated from the TaqMan probe during the PCR cycling. For example, in some embodiments, if the PCR includes 40 cycles, if a signal is generated at any cycle during the amplification, the target gene is considered to be present and detected. In some embodiments, if no signal is generated by the end of the PCR cycling, the target gene is considered to be absent and not detected.

In some embodiments, quantitation of the results of real-time PCR assays is done by constructing a standard curve from a nucleic acid of known concentration and then extrapolating quantitative information for target genes of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate that Ebola is not present in a sample, the Ct values for an endogenous control (such as an SAC) and/or an exogenous control (such as an SPC) must be within a previously-determined valid range. That is, in some embodiments, the absence of Ebola cannot be confirmed unless the controls are detected, indicating that the assay was successful. In some embodiments, the assay includes an exogenous control. In some embodiments, the assay includes an endogenous control. In some embodiments, the assay includes an exogenous control and an endogenous control. Ct values are inversely proportional to the amount of nucleic acid target in a sample.

In some embodiments, a threshold Ct (or a “cutoff Ct”) value for a target gene (including an endogenous control and/or exogenous control), below which the gene is considered to be detected, has previously been determined. In some embodiments, a threshold Ct is determined using substantially the same assay conditions and system (such as a GeneXpert®) on which the samples will be tested.

In addition to the TagMan® assays, other real-time PCR chemistries useful for detecting and quantitating PCR products in the methods presented herein include, but are not limited to, Molecular Beacons, Scorpion probes and intercalating dyes, such as SYBR Green, EvaGreen, thiazole orange, YO-PRO, TO-PRO, etc., which are discussed below.

In various embodiments, real-time PCR detection is utilized to detect, in a single multiplex reaction, the Ebola target genes, and optionally, an endogenous control and/or an exogenous control. In some multiplex embodiments, a plurality of probes, such as TagMan® probes, each specific for a different target, is used. In some embodiments, each target gene-specific probe is spectrally distinguishable from the other probes used in the same multiplex reaction. A nonlimiting exemplary seven-color multiplex system is described, e.g., in Lee et al., BioTechniques, 27: 342-349.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, detection and/or quantitation of real-time PCR products is accomplished using a dye that binds to double-stranded DNA products, such as SYBR Green, EvaGreen, thiazole orange, YO-PRO, TO-PRO, etc. In some embodiments, the analytical method used in the methods described herein is a DASL® (DNA-mediated Annealing, Selection, Extension, and Ligation) Assay. In some embodiments, total RNA is isolated from a sample to be analyzed by any method. Total RNA may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is the same as, or complementary to, at least 8, at least 9, at least 10, at least 11, at least 12, at least 13, at least 14, at least 15, at least 16, at least 17, at least 18, at least 19 contiguous nucleotides of a target RNA, an endogenous control RNA, or an exogenous control RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the target genes in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. See www.luminexcorp.com/technology/index.html. In some embodiments, total RNA is isolated from a sample and is then labeled with biotin. The labeled RNA is then hybridized to target RNA-specific capture probes (e.g., FlexmiR™ products sold by Luminex, Inc. at www.luminexcorp.com/products/assays/index.html) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample is first polyadenylated, and is subsequently labeled with a biotinylated 3DNA™ dendrimer (i.e., a multiple-arm DNA with numerous biotin molecules bound thereto), using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target gene in the methods described herein is by gel electrophoresis and detection with labeled probes (e.g., probes labeled with a radioactive or chemiluminescent label), such as by northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Várallyay, E. et al. (2008) Nature Protocols 3(2):190-196, which is incorporated herein by reference in its entirety.

In some embodiments, detection and quantification of one or more target genes is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra (i.e., detectably different dyes). The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. In some alternative embodiments, a target RNA-specific probe can be labeled with 3 or more distinct labels selected from, e.g., fluorophores, electron spin labels, etc., and then hybridized to an RNA sample.

Optionally, the sample RNA is modified before hybridization. The target RNA/probe duplex is then passed through channels in a microfluidic device and that comprise detectors that record the unique signal of the 3 labels. In this way, individual molecules are detected by their unique signal and counted. See U.S. Pat. Nos. 7,402,422 and 7,351,538 to Fuchs et al., U.S. Genomics, Inc., each of which is incorporated herein by reference in its entirety.

6.2.5. Exemplary Automation and Systems

In some embodiments, gene expression is detected using an automated sample handling and/or analysis platform. In some embodiments, commercially available automated analysis platforms are utilized. For example, in some embodiments, the GeneXpert® system (Cepheid, Sunnyvale, Calif.) is utilized.

The present invention is illustrated for use with the GeneXpert system. Exemplary sample preparation and analysis methods are described below. However, the present invention is not limited to a particular detection method or analysis platform. One of skill in the art recognizes that any number of platforms and methods may be utilized.

The GeneXpert® utilizes a self-contained, single use cartridge. Sample extraction, amplification, and detection may all carried out within this self-contained “laboratory in a cartridge.” (See e.g., U.S. Pat. Nos. 5,958,349, 6,403,037, 6,440,725, 6,783,736, 6,818,185; each of which is herein incorporated by reference in its entirety.)

Components of the cartridge include, but are not limited to, processing chambers containing reagents, filters, and capture technologies useful to extract, purify, and amplify target nucleic acids. A valve enables fluid transfer from chamber to chamber and contain nucleic acids lysis and filtration components. An optical window enables real-time optical detection. A reaction tube enables very rapid thermal cycling.

In some embodiments, the GeneXpert® system includes a plurality of modules for scalability. Each module includes a plurality of cartridges, along with sample handling and analysis components.

After the sample is added to the cartridge, the sample is contacted with lysis buffer and released DNA is bound to a DNA-binding substrate such as a silica or glass substrate. The sample supernatant is then removed and the DNA eluted in an elution buffer such as a Tris/EDTA buffer. The eluate may then be processed in the cartridge to detect target genes as described herein. In some embodiments, the eluate is used to reconstitute at least some of the PCR reagents, which are present in the cartridge as lyophilized particles.

In some embodiments, RT-PCR is used to amplify and analyze the presence of the target genes. In some embodiments, the reverse transcription uses MMLV RT enzyme and an incubation of 5 to 20 minutes at 40° C. to 50° C. In some embodiments, the PCR uses Taq polymerase with hot start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed.

In some embodiments, a double-denature method is used to amplify low copy number targets. A double-denature method comprises, in some embodiments, a first denaturation step followed by addition of primers and/or probes for detecting target genes. All or a substantial portion of the DNA-containing sample (such as a DNA eluate) is then denatured a second time before, in some instances, a portion of the sample is aliquotted for cycling and detection of the target genes. While not intending to be bound by any particular theory, the double-denature protocol may increase the chances that a low copy number target gene (or its complement) will be present in the aliquot selected for cycling and detection because the second denaturation effectively doubles the number of targets (i.e., it separates the target and its complement into two separate templates) before an aliquot is selected for cycling. In some embodiments, the first denaturation step comprises heating to a temperature of 90° C. to 100° C. for a total time of 30 seconds to 5 minutes. In some embodiments, the second denaturation step comprises heating to a temperature of 90° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for a DNA-containing sample (such as a DNA eluate) may comprise heating at least one, at least two, at least three, or at least four aliquots of the sample separately (either sequentially or simultaneously) to a temperature of 90° C. to 100° C. for 60 seconds each. As a non-limiting example, a second denaturation step for a DNA-containing sample (such as a DNA eluate) containing enzyme, primers, and probes may comprise heating at least one, at least two, at least three, or at least four aliquots of the eluate separately (either sequentially or simultaneously) to a temperature of 90° C. to 100° C. for 5 seconds each. In some embodiments, an aliquot is the entire DNA-containing sample (such as a DNA eluate). In some embodiments, an aliquot is less than the entire DNA-containing sample (such as a DNA eluate).

In some embodiments, target genes in a DNA-containing sample, such as a DNA eluate, are detected using the following protocol: One or more aliquots of the DNA-containing sample are heated separately to 95° C. for 60 seconds each. The enzyme and primers and probes are added to the DNA-containing sample and one or more aliquots are heated separately to 95° C. for 5 seconds each. At least one aliquot of the DNA-containing sample containing enzyme, primers, and probes is then heated to 94° C. for 60 seconds. The aliquot is then cycled 45 times with the following 2-step cycle: (1) 94° C. for 5 seconds, (2) 66° C. for 30 seconds.

The present invention is not limited to particular primer and/or probe sequences. Exemplary amplification primers and detection probes are described in the Examples.

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described herein.

6.2.6. Exemplary Data Analysis

In some embodiments, the presence of Ebola is detected if the Ct value for any one of the Ebola target genes (such as the 5′ terminal segment of the Ebola virus) is below a certain threshold. In some embodiments the valid range of Ct values is 12 to 39.9 Ct. In some such embodiments, if no amplification above background is observed from the Ebola-specific primers after 40 cycles, the sample is considered to be negative for Ebola. In some such embodiments, the sample is considered to be negative for Ebola only if amplification of the exogenous control (SPC) and the endogenous control (SAC) (or one of them if only one is used in the assay) are above background.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample or sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence of Ebola) for the subject, with or without recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

6.2.7. Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more nucleotide analogs (i.e., modified nucleotides) discussed herein.

In some embodiments, a polynucleotide is provided that comprises a region that is at least 90%, at least 95%, or 100% identical to, or at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the 5′ terminal segment of the Ebola virus. In some embodiments, a polynucleotide is provided that comprises a region that is at least 90%, at least 95%, or 100% identical to, or complementary to, a span of 6 to 100, 8 to 100, 8 to 75, 8 to 50, 8 to 40, or 8 to 30 contiguous nucleotides of the 5′ terminal segment of the Ebola virus. In some embodiments, the 5′ terminal segment of the Ebola virus is nucleotides 1 to 100 of the Ebola virus. In some embodiments, the 5′ terminal segment of the Ebola virus is nucleotides 1 to 90 of the Ebola virus. In some embodiments, the 5′ terminal segment of the Ebola virus is nucleotides 1 to 80 of the Ebola virus. Nonlimiting exemplary polynucleotides are shown in Table A.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of selectively hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). Thus, in some embodiments, the emission spectrum of the dye should overlap considerably with the absorption spectrum of the quencher. In other embodiments, the dye and quencher are not at the ends of the FRET probe.

6.2.7.1. Exemplary Polynucleotide Modifications

In some embodiments, the methods of detecting at least one target gene described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

6.2.7.2. Exemplary Primers

In some embodiments, a primer is provided. In some embodiments, a primer is at least 80%, at least 85%, at least 90%, at least 95%, or 100% identical to, or at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the 5′ terminal segment of the Ebola virus. In some embodiments, a primer is provided that comprises a region that is at least 80%, at least 85%, at least 90%, at least 95%, or 100% identical to, or complementary to, a span of 6 to 100, 8 to 100, 8 to 75, 8 to 50, 8 to 40, or 8 to 30 contiguous nucleotides of the 5′ terminal segment of the Ebola virus. In some embodiments, the 5′ terminal segment of the Ebola virus is nucleotides 1 to 100 of the Ebola virus. In some embodiments, the 5′ terminal segment of the Ebola virus is nucleotides 1 to 90 of the Ebola virus. In some embodiments, the 5′ terminal segment of the Ebola virus is nucleotides 1 to 80 of the Ebola virus. Nonlimiting exemplary primers are shown in Table A. In some embodiments, a primer may also comprise portions or regions that are not identical or complementary to the target gene. In some embodiments, a region of a primer that is at least 80%, at least 85%, at least 90%, at least 95%, or 100% identical or complementary to a target gene is contiguous, such that any region of a primer that is not identical or complementary to the target gene does not disrupt the identical or complementary region.

In some embodiments, a primer is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, or at least 23 of nucleotides 1 to 23 of the 5′ terminal segment of the Ebola virus. In some embodiments, a primer is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, or at least 23 of nucleotides 1 to 23 of a sequence selected from SEQ ID NOs: 1 to 4. In some embodiments, a primer is at least 80%, at least 85%, at least 90%, at least 95%, or 100% identical to at least 8, at least 9, at least 10, at least 11, at least 12, at least 13, at least 14, at least 15, at least 16, at least 17, at least 18, at least 19, at least 20, at least 21, at least 22, or at least 23 of nucleotides 1 to 23 of a sequence selected from SEQ ID NOs: 1 to 4.

In some embodiments, a primer is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of nucleotides 36 to 79 of the 5′ terminal segment of the Ebola virus. In some embodiments, a primer is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of nucleotides 36 to 79 of a sequence selected from SEQ ID NOs: 1 to 4. In some embodiments, a primer is at least 90%, at least 95%, or 100% identical to at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of nucleotides 36 to 79 of a sequence selected from SEQ ID NOs: 1 to 4.

In some embodiments, a primer comprises a portion that is at least 90%, at least 95%, or 100% identical to a region of a target gene. In some such embodiments, a primer that comprises a region that is at least 90%, at least 95%, or 100% identical to a region of the target gene is capable of selectively hybridizing to a cDNA that has been reverse transcribed from the RNA, or to an amplicon that has been produced by amplification of the target gene. In some embodiments, the primer is complementary to a sufficient portion of the cDNA or amplicon such that it selectively hybridizes to the cDNA or amplicon under the conditions of the particular assay being used.

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. Exemplary hybridization conditions are discussed herein, for example, in the context of a reverse transcription reaction or a PCR amplification reaction. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein.

In some embodiments, a primer comprises a detectable moiety.

In some embodiments, primer pairs are provided. Such primer pairs are designed to amplify a portion of a target gene, such as the 5′ terminal segment of the Ebola virus, or an endogenous control such as a sample adequacy control (SAC), or an exogenous control such as a sample processing control (SPC). In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long. Nonlimiting exemplary primer pairs are shown in Table A.

6.2.7.3. Exemplary Probes

In various embodiments, methods of detecting the presence of Ebola comprise hybridizing nucleic acids of a sample with a probe. In some embodiments, the probe comprises a portion that is complementary to a target gene, such as the 5′ terminal segment of the Ebola virus, or an endogenous control such as a sample adequacy control (SAC), or an exogenous control such as a sample processing control (SPC). In some embodiments, the probe comprises a portion that is at least 80%, at least 85%, at least 90%, at least 95%, or 100% identical to a region of the target gene. In some such embodiments, a probe that is at least 80%, at least 85%, at least 90%, at least 95%, or 100% complementary to a target gene is complementary to a sufficient portion of the target gene such that it selectively hybridizes to the target gene under the conditions of the particular assay being used. In some embodiments, a probe that is complementary to a target gene comprises a region that is at least 80%, at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the target gene. Nonlimiting exemplary probes are shown in Table A. A probe that is at least 80%, at least 85%, at least 90%, at least 95%, or 100% complementary to a target gene may also comprise portions or regions that are not complementary to the target gene. In some embodiments, a region of a probe that is at least 80%, at least 85%, at least 90%, at least 95%, or 100% complementary to a target gene is contiguous, such that any region of a probe that is not complementary to the target gene does not disrupt the complementary region.

In some embodiments, a probe is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of nucleotides 36 to 79 of the 5′ terminal segment of the Ebola virus. In some embodiments, a probe is at least 80%, at least 85%, at least 90%, at least 95%, or 100% identical to, or at least 80%, at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of nucleotides 36 to 79 of a sequence selected from SEQ ID NOs: 1 to 4.

In some embodiments, the probe comprises a portion that is at least 90%, at least 95%, or 100% identical to a region of the target gene, or an endogenous control such as a sample adequacy control (SAC), or an exogenous control such as a sample processing control (SPC). In some such embodiments, a probe that comprises a region that is at least 90%, at least 95%, or 100% identical to a region of the target gene is capable of selectively hybridizing to a cDNA that has been reverse-transcribed from a target gene or to an amplicon that has been produced by amplification of the target gene. In some embodiments, the probe is at least 90%, at least 95%, or 100% complementary to a sufficient portion of the cDNA or amplicon such that it selectively hybridizes to the cDNA or amplicon under the conditions of the particular assay being used. In some embodiments, a probe that is complementary to a cDNA or amplicon comprises a region that is at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the cDNA or amplicon. A probe that is at least 90%, at least 95%, or 100% complementary to a cDNA or amplicon may also comprise portions or regions that are not complementary to the cDNA or amplicon. In some embodiments, a region of a probe that is at least 90%, at least 95%, or 100% complementary to a cDNA or amplicon is contiguous, such that any region of a probe that is not complementary to the cDNA or amplicon does not disrupt the complementary region.

In some embodiments, the method of detecting one or more target genes comprises: (a) reverse transcribing a target RNA to produce a cDNA that is complementary to the target RNA; (b) amplifying the cDNA from (a); and (c) detecting the amount of a target RNA using real time RT-PCR and a detection probe (which may be simultaneous with the amplification step (b)).

As described above, in some embodiments, real time RT-PCR detection may be performed using a FRET probe, which includes, but is not limited to, a TagMan® probe, a Molecular beacon probe and a Scorpion probe. In some embodiments, the real time RT-PCR detection is performed with a TagMan® probe, i.e., a linear probe that typically has a fluorescent dye covalently bound at one end of the DNA and a quencher molecule covalently bound elsewhere, such as at the other end of, the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target gene in the sample is proportional to the amount of fluorescence measured during amplification.

The TagMan® probe typically comprises a region of contiguous nucleotides having a sequence that is at least 90%, at least 95%, or 100% identical or complementary to a region of a target gene or its complementary cDNA that is reverse transcribed from the target RNA template (i.e., the sequence of the probe region is complementary to or identically present in the target RNA to be detected) such that the probe is selectively hybridizable to a PCR amplicon of a region of the target gene. In some embodiments, the probe comprises a region of at least 6 contiguous nucleotides having a sequence that is fully complementary to or identically present in a region of a cDNA that has been reverse transcribed from a target gene. In some embodiments, the probe comprises a region that is at least 90%, at least 95%, or 100% identical or complementary to at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target gene to be detected.

In some embodiments, the region of the amplicon that has a sequence that is at least 90%, at least 95%, or 100% complementary to the TagMan® probe sequence is at or near the center of the amplicon molecule. In some embodiments, there are independently at least 2 nucleotides, such as at least 3 nucleotides, such as at least 4 nucleotides, such as at least 5 nucleotides of the amplicon at the 5′-end and at the 3′-end of the region of complementarity.

In some embodiments, Molecular Beacons can be used to detect PCR products. Like TagMan® probes, Molecular Beacons use FRET to detect a PCR product via a probe having a fluorescent dye and a quencher attached at the ends of the probe. Unlike TagMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see www.genelink.com/newsite/products/mbintro.asp).

In some embodiments, Scorpion probes can be used as both sequence-specific primers and for PCR product detection. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached elsewhere, such as to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g., Premier Biosoft International (see www.premierbiosoft.com/tech_notes/Scorpion.html).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye′; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction.

Specific examples of fluorescently labeled ribonucleotides useful in the preparation of PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target genes are detected in a single multiplex reaction. In some such embodiments, each probe that is targeted to a unique amplicon is spectrally distinguishable when released from the probe, in which case each target gene is detected by a unique fluorescence signal. In some embodiments, two or more target genes are detected using the same fluorescent signal, in which case detection of that signal indicates the presence of either of the target genes or both.

One skilled in the art can select a suitable detection method for a selected assay, e.g., a real-time RT-PCR assay. The selected detection method need not be a method described above, and may be any method.

### 6.3. Exemplary Compositions and Kits

In another aspect, compositions are provided. In some embodiments, compositions are provided for use in the methods described herein.

In some embodiments, compositions are provided that comprise at least one target gene-specific primer. The terms “target gene-specific primer” and “target RNA-specific primer” are used interchangeably and encompass primers that have a region of contiguous nucleotides having a sequence that is (i) at least 90%, at least 95%, or 100% identical to a region of a target gene, or (ii) at least 90%, at least 95%, or 100% complementary to the sequence of a region of contiguous nucleotides found in a target gene. In some embodiments, a composition is provided that comprises at least one pair of target gene-specific primers. The term “pair of target gene-specific primers” encompasses pairs of primers that are suitable for amplifying a defined region of a target gene. A pair of target gene-specific primers typically comprises a first primer that comprises a sequence that is at least 90%, at least 95%, or 100% identical to the sequence of a region of a target gene and a second primer that comprises a sequence that is at least 90%, at least 95%, or 100% complementary to a region of a target gene. A pair of primers is typically suitable for amplifying a region of a target gene that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 50 to 100 nucleotides long, 50 to 90 nucleotides long, 50 to 80 nucleotides long, 60 to 80 nucleotides long, 100 to 300 nucleotides long, 100 to 200 nucleotides long, or 100 to 150 nucleotides long. Nonlimiting exemplary primers, and pairs of primers, are shown in Table A.

In some embodiments, a composition comprises at least one pair of target gene-specific primers. In some embodiments, a composition additionally comprises a pair of target gene-specific primers for amplifying an endogenous control (such as an SAC) and/or one pair of target gene-specific primers for amplifying an exogenous control (such as an SPC).

In some embodiments, a composition comprises at least one target gene-specific probe. The terms “target gene-specific probe” and “target RNA-specific probe” are used interchangeably and encompass probes that have a region of contiguous nucleotides having a sequence that is (i) at least 90%, at least 95%, or 100% identical to a region of a target gene, or (ii) at least 90%, at least 95%, or 100% complementary to the sequence of a region of contiguous nucleotides found in a target gene. Nonlimiting exemplary target-specific probes are shown in Table A.

In some embodiments, a composition (including a composition described above that comprises one or more pairs of target gene-specific primers) comprises one or more probes for detecting the target genes. In some embodiments, a composition comprises a probe for detecting an endogenous control (such as an SAC) and/or a probe for detecting an exogenous control (such as an SPC).

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include one or more buffering components and/or additional components.

In some embodiments, a composition further comprises one or more additional components. Additional components include, but are not limited to, salts, such as NaCl, KCl, and MgCl₂; polymerases, including thermostable polymerases such as Taq; dNTPs; reverse transcriptases, such as MMLV reverse transcriptase; Rnase inhibitors; bovine serum albumin (BSA) and the like; reducing agents, such as β-mercaptoethanol; EDTA and the like; etc. One skilled in the art can select suitable composition components depending on the intended use of the composition.

In some embodiments, compositions are provided that comprise at least one polynucleotide for detecting at least one target gene. In some embodiments, the polynucleotide is used as a primer for a reverse transcriptase reaction. In some embodiments, the polynucleotide is used as a primer for amplification. In some embodiments, the polynucleotide is used as a primer for PCR. In some embodiments, the polynucleotide is used as a probe for detecting at least one target gene. In some embodiments, the polynucleotide is detectably labeled. In some embodiments, the polynucleotide is a FRET probe. In some embodiments, the polynucleotide is a TagMan® probe, a Molecular Beacon, or a Scorpion probe.

In some embodiments, a composition comprises at least one FRET probe having a sequence that is at least 90%, at least 95%, or 100% identical, or at least 90%, at least 95%, or 100% complementary, to a region of, a target gene, such as the 5′ terminal segment of the Ebola virus. In some embodiments, a FRET probe is labeled with a donor/acceptor pair such that when the probe is digested during the PCR reaction, it produces a unique fluorescence emission that is associated with a specific target gene. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target gene. In some embodiments, the sequence of the FRET probe is complementary to a target region of a target gene. In other embodiments, the FRET probe has a sequence that comprises one or more base mismatches when compared to the sequence of the best-aligned target region of a target gene.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is at least 90%, at least 95%, or 100% identical, or at least 90%, at least 95%, or 100% complementary, to a region of, a target gene, such as the 5′ terminal segment of the Ebola virus. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, a target gene, such as the 5′ terminal segment of the Ebola virus. In some embodiments, the FRET probe has a sequence with one, two or three base mismatches when compared to the sequence or complement of the target gene.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target gene-specific FRET probes and/or one or more primers for reverse transcription of target RNAs and/or one or more primers for amplification of target genes or cDNAs reverse transcribed therefrom.

In some embodiments, one or more of the primers and/or probes is “linear”. A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target gene.

In some embodiments, a kit comprises one or more pairs of linear primers (a “forward primer” and a “reverse primer”) for amplification of a target gene or cDNA reverse transcribed therefrom. Accordingly, in some embodiments, a first primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 90%, at least 95%, or 100% identical to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a first location in the target gene. Furthermore, in some embodiments, a second primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 90%, at least 95%, or 100% complementary to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a second location in the target gene, such that a PCR reaction using the two primers results in an amplicon extending from the first location of the target gene to the second location of the target gene.

In some embodiments, the kit comprises at least two, at least three, or at least four sets of primers, each of which is for amplification of a different target gene or cDNA reverse transcribed therefrom. In some embodiments, the kit further comprises at least one set of primers for amplifying a control RNA, such as an endogenous control and/or an exogenous control.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the kits for use in real time RT-PCR methods described herein further comprise reagents for use in the reverse transcription and amplification reactions. In some embodiments, the kits comprise enzymes, such as a reverse transcriptase or a heat stable DNA polymerase, such as Taq polymerase. In some embodiments, the kits further comprise deoxyribonucleotide triphosphates (dNTP) for use in reverse transcription and/or in amplification. In further embodiments, the kits comprise buffers optimized for specific hybridization of the probes and primers.

A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the methods described herein. Instructions included in kits can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit can comprise the reagents described above provided in one or more GeneXpert® cartridge(s). These cartridges permit extraction, amplification, and detection to be carried out within this self-contained “laboratory in a cartridge.” (See e.g., U.S. Pat. Nos. 5,958,349, 6,403,037, 6,440,725, 6,783,736, 6,818,185; each of which is herein incorporated by reference in its entirety.) Reagents for measuring genomic copy number level and detecting a pathogen could be provided in separate cartridges within a kit or these reagents (adapted for multiplex detection) could be provide in a single cartridge.

Any of the kits described here can include, in some embodiments, a receptacle for a nasal aspirate/wash sample and/or a swab for collecting a nasopharyngeal swab sample.

The following examples are for illustration purposes only, and are not meant to be limiting in any way.

### 6.4. Example 1

### Detection of Ebola

A systematic search for segments of around 100 nucleotides of the Ebola virus that are highly conserved across all five subtypes (Zaire, Bundibugyo, Tai Forest, Sudan, and Reston) was carried out. While some segments are conserved between certain pairs of genomes, no segments are conserved across all five genomes, except the 5′ end of the genomes, upstream of the NP gene.

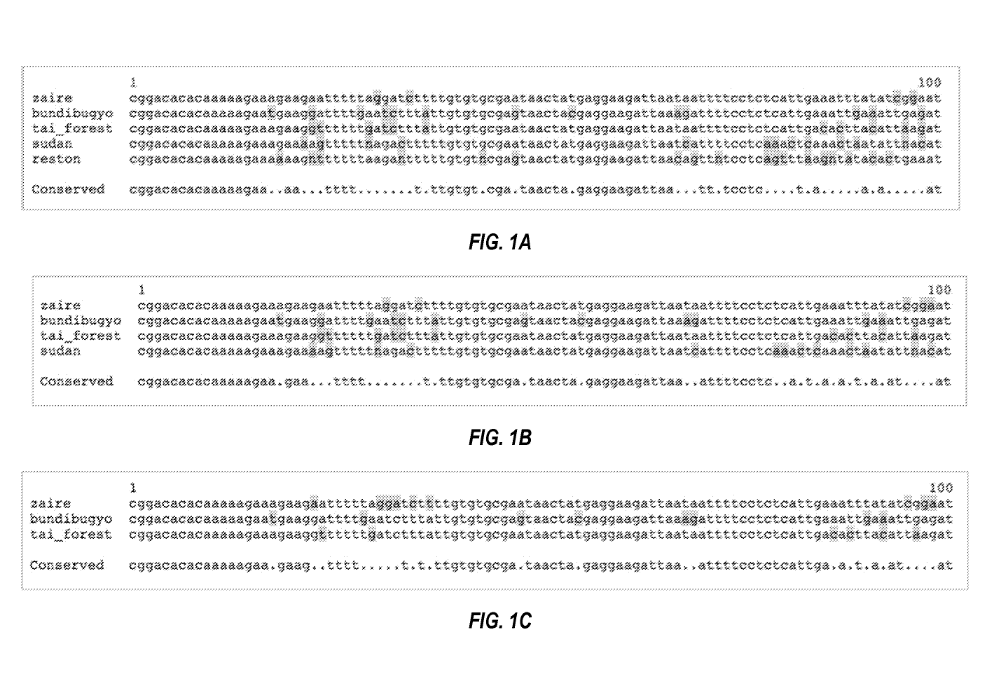

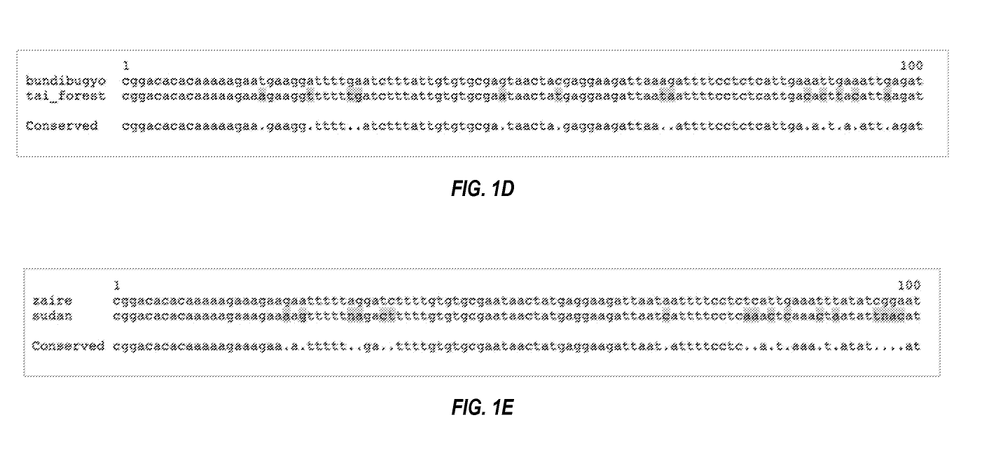

FIG. 1A shows an alignment of nucleotides 1 to 100 of Ebola vim subtypes (or “strains”) Zaire, Bundibugyo, Tai Forest, Sudan, and Reston. An “n” in the nucleotide sequence represents a position with some variation in the alignment of all genomes for that subtype. Shaded positions are those that differ from the consensus of the current alignment. The alignment shows complete conservation in the 5′ 17 nucleotides followed by shorter blocks of complete conservation within the first 80 nucleotides.

Since the Reston subtype of Ebola virus is not known to be infectious in humans, an alignment of the other four subtypes in this region was constructed, and is shown in FIG. 1B. Higher conservation is observed between the Zaire, Bundibugyo, Tai Forest, and Sudan strains of Ebola virus.

Alignments of the 5′ terminal segment of three strains (Zaire, Bundibugyo, and Tai Forest), and pairs of strains (Bundibugyo and Tai Forest, and Zaire and Sudan) were also constructed, and are shown in FIGS. 1C to 1E. The degree of conservation between the sequences increases when fewer strains are included in the alignment. The degree of conservation between at least the four strains, Zaire, Bundibugyo, Tai Forest, and Sudan, suggests that primers and probes may be designed that can detect all four strains. In some cases, limited degenerate nucleotide positions may be used in one or more of the primers and probe.

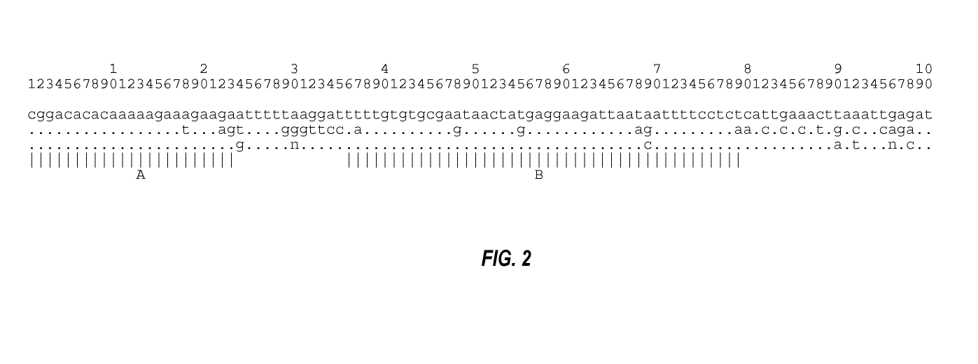

FIG. 2 is a different representation of the consensus from FIG. 1B. The top line shows the consensus sequence, using the most common nucleotide at each position. For non-conserved positions, the second and third lines show the alternate nucleotides found at that position. Finally, the vertical lines indicate segments that are particularly well conserved and therefore are good targets for primer design. Segment A is nucleotides 1 to 23 and segment B is nucleotides 36 to 79.

From the segments identified in FIG. 2, forward primers were designed to anneal to segment A and reverse primers and probes were designed to anneal to segment B. Table A shows the sequences of the primers and probe. Nucleotides in parentheses indicate degenerate positions; a mixture of primers with the the indicated nucleotides at that position is used. The sequences, showing the degenerate positions using IUPAC nucleotide codes, are also shown in the Table of Certain Sequences herein. The primers produce an amplicon that is 77 nucleotides long.

TABLE A
Primers and Probes for Detecting Ebola 5′ Terminal Segment

| Description | Sequence | SEQ ID NO |
|---|---|---|
| 5′ terminal segement | gacacacaaaaagaa(a/t)g | 6 |
| forward primer | | |
| 5′ terminal segement | (a/t)gaggaaaat(t/a/g)(a/t)ttaatctt | 7 |
| reverse primer | | |
| 5′ terminal segement | F1-ttgtgtgcga(a/g)taacta(t/c)gagg -Q1 | 8 |
| probe | | |

F1, F2, and F3 are detectably different dyes that can be detected and distinguished simultaneously in a multiplex reaction. Each probe also comprises a quencher (e.g., Q1 or Q2). In some embodiments, if more than one Ebola gene is detected in a single multiplex assay, the probes for the Ebola genes may comprise the same or different dyes.

Each reaction contains 100 nM to 800 nM of each probe and primer, 50-90 mM KCl, 3-5 mM MgCl₂, 400-825 μM dNTPs, 20 mM Tris, pH 8.5, 0.01% sodium azide, and 1 units/μl of RNase inhibitor. MMLV reverse transcriptase (2 units/μl) and AptaTaq (3 units/μl; Roche) are used for reverse transcription and amplification, respectively.

One ml of buffer containing cells from a patient sample (e.g., oral swab or blood) are loaded into a GeneXpert® cartridge for analysis. The sample is mixed with a lysis reagent to release nucleic acids. After lysis, the released nucleic acid from the sample are captured on a nucleic acid-binding substrate. The nucleic acid is eluted from the substrate and used to reconstitute the reagents used for real-time PCR (described above). An exemplary reaction cycle is: 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. using a GeneXpert® cartridge in a GeneXpert® system.

The valid range of Ct values for the targets is 12-39.9 Ct.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

TABLE OF CERTAIN SEQUENCES

| SEQ ID | | |
|---|---|---|
| NO | Description | Sequence |
| 1 | Ebola virus, Zaire | CGGACACACA AAAAGAAAGA AGAATTTTTA GGATCTTTTG |
| | strain, the 5′ | TGTGCGAATA ACTATGAGGA AGATTAATAA TTTTCCTCTC |
| | terminal segment | ATTGAAATTT ATATCGGAAT |
| 2 | Ebola virus, | CGGACACACA AAAAGAATGA AGGATTTTGA ATCTTTATTG |
| | Bundibugyo | TGTGCGAGTA ACTACGAGGA AGATTAAAGA TTTTCCTCTC |
| | strain, the 5′ | ATTGAAATTG AAATTGAGAT |
| | terminal segment | |
| 3 | Ebola virus, Tai | CGGACACACA AAAAGAAAGA AGGTTTTTTG ATCTTTATTG |
| | Forest strain, the | TGTGCGAATA ACTATGAGGA AGATTAATAA TTTTCCTCTC |
| | 5′ terminal | ATTGACACTT ACATTAAGAT |
| | segment | |
| 4 | Ebola virus, | CGGACACACA AAAAGAAAGA AAAGTTTTTN AGACTTTTTG |
| | Sudan strain, the | TGTGCGAATA ACTATGAGGA AGATTAATCA TTTTCCTCAA |
| | 5′ terminal | ACTCAAACTA ATATTNACAT |
| | segment | |
| 5 | Ebola virus, | CGGACACACA AAAAGAAAAA AGNTTTTTTA AGANTTTTTG |
| | Reston strain, the | TGTNCGAGTA ACTATGAGGA AGATTAACAG TTNTCCTCAG |
| | 5′ terminal | TTTAAGNTAT ACACTGAAAT |
| | segment | |
| 6 | 5′ terminal | GACACACAAAAAGAAWG |
| | segement | W = A OR T |
| | forward primer | |
| 7 | 5′ terminal | WGAGGAAAATVWTTAATCTT |
| | segement | W = A OR T; V = A, C, OR G |
| | reverse primer | |
| 8 | 5′ terminal | TTGTGTGCGARTAACTAYGAGG |
| | segement probe | R = A OR G; Y = C OR T |

1. A method of detecting the presence or absence of Ebola virus in a sample from a subject comprising detecting the presence or absence of a 5′ terminal segment of the Ebola virus.

2. The method of claim 1, wherein the Ebola virus is a subtype selected from Zaire, Bundibugyo, Cote D'Ivoire, Tai Forest, Sudan, and Reston.

3. (canceled)

4. The method of claim 1, wherein the 5′ terminal segment of the Ebola virus is nucleotides 1 to 100.

5. The method of claim 1, wherein the 5′ terminal segment of the Ebola virus is at least 80%, at least 85%, at least 90% identical to a sequence selected from SEQ ID NOs: 1 to 5.

6-23. (canceled)

24. The method of claim 1, wherein the method comprises PCR.

25. (canceled)

26. (canceled)

27. The method of claim 1, wherein the method comprises contacting nucleic acids from the sample with a first primer pair for detecting the 5′ terminal segment of the Ebola virus.

28-41. (canceled)

42. The method of claim 1, wherein the method comprises contacting the sample with an Ebola 5′ terminal segment probe comprising a detectable label.

43-48. (canceled)

49. The method of claim 1, wherein the method comprises detecting the presence of absence of the 5′ terminal segment of the Ebola virus, and no other Ebola genes.

50. (canceled)

51. A composition comprising a first primer pair for detecting the 5′ terminal segment of the Ebola virus.

52-75. (canceled)

76. A kit comprising a composition of claim 51.

77. The kit of claim 76, wherein the kit further comprises an exogenous control.

78. The kit of claim 77, wherein the exogenous control is an Armored® RNA.

79. (canceled)

80. (canceled)

81. An oligonucleotide consisting of a sequence selected from SEQ ID NOs: 6 to 8, wherein the oligonucleotide comprises at least one modified nucleotide.

82. The oligonucleotide of claim 81, wherein the oligonucleotide comprises a detectable label.

83. The oligonucleotide of claim 82, wherein the oligonucleotide comprises a fluorescent dye and a quencher molecule.

84. (canceled)

85. A composition comprising at least one set of primers comprising a first primer consisting of the sequence of SEQ ID NO: 6 and a second primer consisting of the sequence of SEQ ID NO: 7, wherein at least one primer in the composition comprises at least one modified nucleotide.

86. The composition of claim 85, wherein the composition comprises a probe consisting of the sequence of SEQ ID NOs: 8, wherein the probe comprises at least one modified nucleotide and/or a detectable label.

87-88. (canceled)

89. The composition of claim 85, wherein the probe comprises at least one modified nucleotide.

90. The composition of claim 85, wherein the composition is a lyophilized composition.

91. The composition of claim 85, wherein the composition is in solution.

92. (canceled)
